(19) United States
(12) Patent Application Publication
(10) Pub. No.: US 2025/0257373 A1
(43) Pub. Date: Aug. 14, 2025

(54) BIO-OXIDATION PROCESS FOR THE PREPARATION OF ALKANEDIOIC ACIDS

(71) Applicant: ExxonMobil Technology and Engineering Company, Spring, TX (US)

(72) Inventors: Xiaozhou Zhang, Bridgewater, NJ (US); Jorg F. W. Weber, Houston, TX (US); Yunlong Zhang, Cypress, TX (US)

(21) Appl. No.: 19/041,444

(22) Filed: Jan. 30, 2025

Related U.S. Application Data
(60) Provisional application No. 63/552,362, filed on Feb. 12, 2024.

(51) Int. Cl. C12P 7/46; C07C 67/48; C11C 3/00; C12P 7/40

(52) U.S. Cl. CPC C12P 7/46; C07C 67/48; C11C 3/003; C12P 7/40

(57) ABSTRACT

Methods for bio-oxidation of α-alkenoic acids or esters to prepare the corresponding α,ω-alkanedioic acids or esters is disclosed. In an embodiment, the present bio-oxidation methods convert a co-product, 9-DAME, of the ethenolysis of methyl oleate to produce sebacic acid under environmentally friendly conditions.

### CROSS-REFERENCE TO RELATED APPLICATIONS

This application claims the benefit of U.S. Provisional Application No. 63/552,362, filed on Feb. 12, 2024, the entire contents of which is incorporated herein by reference.

### FIELD

The present disclosure generally relates to bio-oxidation processing of α,ω-alkanedioic acids and esters, and more specifically to the bioconversion of decanoic acid methyl ester to produce decanedioic acid (sebacic acid), a co-product of the ethenolysis of oleochemicals such as vegetable oils.

### BACKGROUND

Biocatalysis is an enabling technology that offers an alternative entry-point to selective partial oxidation of medium- and long-chain hydrocarbons. Often biocatalytic oxidations can achieve regio- and stereo-selectivity that are not feasible with chemical catalysis. Renewable feedstocks include biomass. Biomass are materials derived from living organisms, usually plants such as vegetable oils and other oils comprising fatty acid methyl esters. Notwithstanding recent advances, technology enabling conversion from biomass into bio-based chemicals and into final products that people use is not yet well developed.

For example, ethenolysis can produce a commercially significant linear alpha-olefin, 1-decene which has applications that include base stocks. A co-product of an ethenolysis process of methyl oleate is decanoic acid methyl ester that requires additional processing that is not environmentally friendly and/or cost effective.

Therefore, bioconversion alternatives such as production of α,ω-alkanedioic acids by bio-oxidation with Candidas tropicalis strains have been proposed. As reported, dodecane was found to be toxic to a yeast Candida sp. bio-oxidation reaction, limiting its utility in the preparation of α,ω-alkanedioic acids and to provide an integrated bioconversion process without costly and environmentally unfriendly downstream processing of the co-product.

### SUMMARY

Provided herein are methods of preparing α,ω-alkanedioic acids or esters of Formula I.

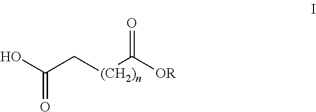

where n≥1 and R is H, C₁-C₁₈ alkyl, optionally substituted C₂-C₅ alkenyl, C₃-C₇ cycloalkyl, acetol, 2-N-(morpholinyl)ethyl, (methoxyethoxy)ethyl, methoxyethyl, cholie, substituted methyl, 2-substituted ethyl, optionally substituted phenyl, optionally substituted benzyl, optionally substituted phenacyl, or silyl comprising reacting a compound of Formula II:

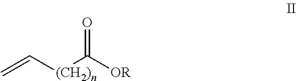

where n≥1 and R is H, C₁-C₁₈ alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl with a bio-oxidation agent.

Also provided are methods of preparing α,ω-alkanedioic acids of Formula III:

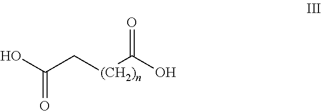

where n≥1 comprising
a) reacting a compound of Formula IV:

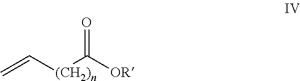

where n≥1 and R′ is C₁-C₁₈ alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl with a bio-oxidation agent to provide a compound of Formula V:

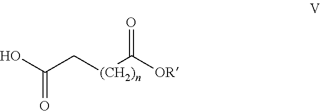

and
b) cleaving the ester moiety.

As described herein, the bio-oxidation agent is one or more naturally occurring or modified yeast, including Saccharomyces cerevisae, Yarrowia lipolytica, Yarrowia galli, Yarrowia oslonensis, Scheffersomyces stipitis, Candida parapsilosis, Candida maltosa, Candida viswanathii, Candida psuedoglaebosa, Meyerozyma guilliermondii, Debaryomyces hansenii, Candida tropicalis, Lodderomyces. elongisporus, Diutina catenulata, Yarrowia deformans, Candida phangneansis, Kodamaea ohmeri, Starmerella apicola, Starmerella bombicola, Candida krusei, Pichia fermentans, Candida alimentaria, Candida hispaniensis, and Candida hollandica.

The bio-oxidation agent is one or more bacteria including Pseudomonas putida, alcanivorax borkumensis, rhodococcus sp., acineobacter, mycobacterium, burkholderia, marinobacter, gordonia sp., dietzia sp., nocardia sp., geobacillus sp., Pseudomonas aeruginosa, bacillus subtillus, arthrobacter sp., thermus sp., achromobacter sp., and sphingomonas sp.

These and other features and attributes of the disclosed methods of the present disclosure and their advantageous applications and/or uses will be apparent from the detailed description which follows.

### BRIEF DESCRIPTION OF DRAWINGS

To assist those of ordinary skill in the relevant art in making and using the subject matter hereof, reference is made to the appended drawings, wherein:

FIG. 1 depicts a vertically-integrated process that converts a vegetable oil, a renewable feedstock, comprising a fatty acid methyl ester into 1-decene and sebacic acid without a downstream process having a detrimental environmental impact and/or without caustic decomposition of ricinoleic acid.

FIG. 2A is a base peak chromatogram (BPC) for the bioconversion of methyl dec-9-enoate.

FIG. 2B is a base peak chromatogram (BPC) for the bioconversion of decanoic acid.

FIG. 2C is a base peak chromatogram (BPC) for the bioconversion of methyl decanoate.

FIG. 3A is an extracted ion chromatograph (EIC) of the feed in the bioconversion of decanoic acid.

FIG. 3B is an extracted ion chromatograph (EIC) of the diacid product in the bioconversion of decanoic acid.

FIG. 3C is an extracted ion chromatograph (EIC) of the intermediate in the bioconversion of decanoic acid.

FIG. 4 is an extracted ion chromatograph (EIC) of decanedioic acid (product) for the bioconversion of dec-9-enoic acid.

### DETAILED DESCRIPTION

The general chemical terms in the Formula above have their usual meanings. For example, the term “C₁-C₁₈ alkyl” includes linear and all branched C₁-C₁₈ alkyl moieties such as methyl, ethyl, all isomers of propyl including n-propyl and isopropyl, all isomers of butyl including n-butyl, isobutyl, sec-butyl, and tert-butyl, all isomers of pentyl, all isomers of hexyl, all isomers of heptyl, and all isomers of octyl, all isomers of nonyl, all isomers of decyl, all isomers of undecanyl, all isomers of dodecanyl, all isomers of tridecanyl, all isomers of tetradecanyl, all isomers of pentadecanyl, all isomers of hexadecanyl, all isomers of heptadecanyl, all isomers of ocatadecanyl. and the like. The term “C₁-C₄ alkyl” includes the linear and all branched C₁-C₄ alkyl moieties such as methyl, ethyl, propyl, isopropyl, butyl, isobutyl, sec-butyl, tert-butyl, and the like.

The term “C₃-C₇ cycloalkyl” includes cyclopropyl, cyclobutyl, cyclopentyl, cyclohexyl, and cycloheptyl.

The term “C₁-C₄ alkoxy” includes methoxy, ethoxy, propoxy, isopropoxy, butoxy, and the like.

The term “C₁-C₄ alkylthio” includes methylthio, ethylthio, propylthio, isopropylthio, butylthio, and the like.

The term “halo” includes fluoro, chloro, bromo, and iodo.

The term “optionally substituted C₂-C₅ alkenyl” includes linear and branched isomers of alkenes including ethenyl, propenyl, butenyl, 2-methylbut-3-en-2-yl, and pentenyl and the like optionally substituted at least once with halo.

The term “substituted methyl” includes 9-fluorenylmethyl, methoxymethyl, methoxyethoxymethyl, methylthiomethyl, tetrahydropyranyl, tetrahydrofuranyl, 2-(trimethylsilyl)ethoxymethyl, benzyloxymethyl, triisopropylsilyloxymethyl, pivaloyloxymethyl, phenylacetoxymethyl, triisopropylsilylmethyl, dicyclopropylmethyl, cyanomethyl, and the like.

The term “2-substituted ethyl” includes an ethyl group substituted at the 2-position with C₁-C₄ alkoxy, C₁-C₄ alkylthio, cyano, or 1-3 times at the 2-position with halo, and the like.

The term “optionally substituted phenacyl” includes phenacyl and phenacyl substituted on the phenyl ring with 1 to 3 substituents selected from halo, hydroxy, C₁-C₄ alkyl, C₁-C₄ alkoxy, and the like.

The term “optionally substituted phenyl” includes phenyl, pentafluorophenyl, and a phenyl group substituted with 1 to 3 substituents such as C₁-C₄ alkyl, C₁-C₄ alkoxy, C₁-C₄ alkylthio, and the like.

The term “optionally substituted benzyl” includes benzyl and benzyl substituted with 1-3 substituents such as halo, nitro, C₁-C₄ alkyl, C₁-C₄ alkoxy, and the like.

The term “silyl” includes trimethylsilyl, triethylsilyl, tert-butyldimethylsilyl, tert-butyldiphenylsilyl, isopropyldimethylsilyl, phenyldimethylsilyl, and di-(tert-butyl)methylsilyl, and the like.

Market demand for linear alpha olefins (“LAOs”) has been increasing in recent years, as LAOs can be used to make many valuable products including plastics, polymers, and synthetic lubricants. The most desired LAOs have carbon chain lengths of C₄ to C₁₄. Processes for making C₄ to C₁₄ LAOs, however, also inevitably produce C₁₂ to C₂₆₊ LAOs, leading to a surplus of these longer-chain LAOs. As such, there is an interest in alternative and sustainable methods for oxyfunctionalization and low-carbon processes that can upgrade long-chain LAOs to higher-value chemicals.

For example, bio-based 1-decene can be produced through the ethenolysis of methyl oleate with homogeneous ruthenium olefin metathesis catalysts to form 1-decene and co-product 9-decenoic acid methyl ester (“9-DAME”). To make the ethenolysis of 1-decene economic, 9-DAME, must be upgraded. Options to upgrade 9-DAME include conversions to 1-decanol, decanoic acid, 1-decene, and sebacic acid, a high value product. Applications of sebacic acid include monomeric and polymeric plasticizers for PVC, diesters for engine lubricants, and polymer intermediates for polyamides, polyesters, and polyurethane adhesives, coatings, fibers, inks, and resins. The low toxicity of the sebacate esters makes them especially useful in food packaging films and plastics. Polyesters from glycols and sebacic acid yield nonmigrating plasticizers. Currently, sebacic acid is manufactured by the caustic decomposition of ricinoleic acid (obtained from castor oil). The process heavily relies on the limited single source of feedstock castor oil and exhibits high environmental impact.

Bio-based 1-decene produced through the ethenolysis of methyl oleate with homogeneous ruthenium olefin metathesis catalysts forms 1-decene and a co-product, 9-DAME). As described herein, the co-product 9-DAME can be converted with a bio-oxidation agent (such as yeast) to produce sebacic acid, making the production of 1-decene via biocatalytic oxidation economically feasible. Optionally, the present methods provide a vertically-integrated process for converting a renewable feedstock into a high quality base stock and a valued co-product sebacic acid without a process that negatively impacts the environment and/or without caustic decomposition of ricinoleic acid.

As noted above, the ethenolysis of an olefin to produce LAOs has a commercial interest. LAOs are useful as monomers or comonomers to produce polyalphaolefins (“PAOs”) and/or as intermediates in the production of epoxides, amines, oxo alcohols, synthetic lubricants, synthetic fatty acids, and alkylated aromatics. LAOs of industrial importance include 1-butene, 1-hexene, 1-octene, 1-decene, 1-undecene, 1-dodecene, and 1-tetradecene. However, the production of LAOs is often undesirably inefficient, creates unwanted by-products, and wastes reactants and energy. Also, the major source of the starting materials for these commercial routes to LAOs is a nonrenewable feedstream including petroleum, coal, and natural gas.

Recently there has been a strong incentive to produce fuels and chemical products from renewable feedstreams such as natural oils. For example, the development of biodiesel fuels is of great interest and some biodiesel-based materials are already commercially produced. Specifically, demand for bio-diesel fuels made from plant oils is expected to increase significantly over the next decade. LAOs such as 1-decene are available from such renewable feedstreams by a CM reaction of the renewable feedstream, such as methyl oleate, with an olefin, such as ethylene, in the presence of a metathesis catalyst. 1-Decene is used to produce polyalphaolefin synthetic lubricant base-stock and surfactants.

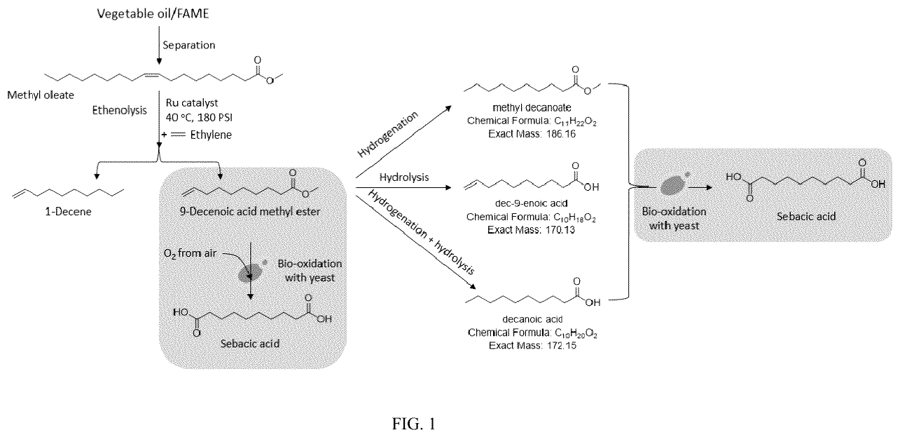

As depicted in FIG. 1, CM catalysts for the ethenolysis of methyl oleate are typically ruthenium-based catalysts bearing phosphine or carbene ligands. WO 2008/010961; Burdett, K., et al., Renewable Monomer Feedstocks via Olefin Metathesis: Fundamental Mechanistic Studies of Methyl Oleate Ethenolysis with the First-Generation Grubbs Catalyst, Organometallics 23, 9, 2027-2047 (2004). Because of cost and safety concerns associated with industrial scale reactions comprising diazo compounds have led to increased efforts to prepare ruthenium alkylidenes via alternate synthetic routes, such as using propargyl and vinyl chlorides. Wilhelm, T. et al., Reactivity of Ru(H)(H₂)Cl(PCy₃)₂ with Propargyl and Vinyl Chlorides: New Methodology to Give Metathesis-Active Ruthenium Carbene, Organometallics, 16, 3867-3869 (1997). Further, to obtain a commercially viable metathesis-based process, for example, LAO production (producing 1-decene) via the CM of ethylene and biodiesel or natural oils, high activity metathesis catalysts have been discovered. See e.g., U.S. Pat. No. 8,524,930.

Aliphatic α,ω-dicarboxylic acids are used in the synthesis of a variety of chemicals important to the production of pharmaceuticals, plastics, perfumes, plasticizers, and greases. They are often produced from natural products under harsh chemical oxidation conditions employing, for example, nitric acid, potassium permanganate or chromic acid, or by treatment with strong base, all of which carry a high environmental impact.

The preparation of malonic acid, succinic acid, glutaric acid, and adipic acid by microbial fermentation using genetically modified Escherichia coli, Mannheimia succiniciproducens, or Corynebacterium glutamicum using one or both of glucose and glycerol as a carbon source has been reported. See, Ji Yeon Kim, et al., Recent Advances in the Production of Platform Chemicals Using Metabolically Engineered Microorganisms, Current Opinion in Green and Sustainable Chemistry, 40, 100777 (2023); Tong Un Chae, et al., Metabolic Engineering for the Production of Dicarboxylic Acids and Diamines, Metabolic Engineering, 58, 2 (2020). The production of α,ω-alkanedioic acids by bio-oxidation of alkanes and fatty acids with Candidas tropicalis strains have also been described. See e.g., U.S. Pat. No. 5,254,466. However, the terminal olefin of 1-dodecene has been taught to be toxic to a yeast Candida sp. bio-oxidation reaction, limiting its utility in the preparation of α,ω-alkanedioic acids. Bioconversion of Linear Alpha Olefin, ip.com/IPCOM/000263765, Oct. 2, 2020.

Therefore, provided herein are methods for an environmentally friendly, economic bio-oxidation method to prepare sebacic acid and other α,ω-alkanedioic acids or esters from the corresponding α-alkenoic acids or esters.

### Bio-Oxidation Reaction

The present methods are drawn to the bio-oxidation of α-alkenoic acids or esters of Formula II to provide the compounds of Formula I as illustrated in Scheme 1 where variables “R” and “n” are as previously defined:

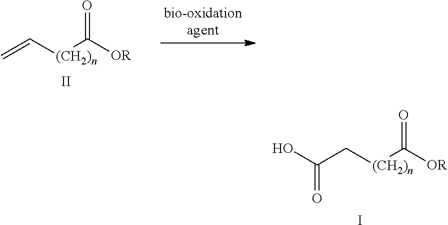

The α-alkenoic acids or esters of Formula II are either commercially available or may be prepared by methods well known to the skilled artisan. Examples of known procedures and methods useful for the preparation of compounds of Formula II include those described in general reference texts such as Comprehensive Organic Transformations, VCH Publishers Inc, 1989; Compendium of Organic Synthetic Methods, Volumes 1-10, 197 4-2002, Wiley Interscience; Advanced Organic Chemistry, Reactions Mechanisms, and Structure, 5th Edition, Michael B. Smith and Jerry March, Wiley Interscience, 2001; Advanced Organic Chemistry, 4th Edition, Part B, Reactions and Synthesis, Francis A Carey and Richard J. Sundberg, Kluwer Academic/Plenum Publishers, 2000, and references cited therein. Specific methods for preparing esters from carboxylic acids are described in Greene's Protective Groups in Organic Synthesis, Fifth Edition, by Peter G. M. Wuts and Theodora W. Greene, John Wiley and Sons, Inc., 692-699 (2014), hereinafter “Greene.”

### Growth Phase

The bio-oxidation process occurs in a suitable reaction medium. Suitable reaction media include buffered glycerol-complex medium and buffered methanol-complex medium. The reaction medium depends upon the requirements of the bio-oxidation agent and the properties of the α-alkenoic acid or ester. A first reaction medium is inoculated with colonies of the desired bio-oxidation agent and these colonies are allowed to grow. The pH of the reaction medium for this growth phase is maintained in the range of 3-5. The temperature of the reaction medium during the growth phase depends upon the bio-oxidation agent employed but is between 2° and 40° C. The length of the growth phase depends upon the bio-oxidation agent employed and is from 8 hours to 24 hours.

### Bio-Oxidation Phase

After the growth phase, a portion of the first reaction medium containing the bio-oxidation agent is added to a second reaction medium containing the compound of Formula II. This second reaction medium may be the same as or different than the first reaction medium. The concentration of the compound of Formula II will vary depending upon the requirements of the bio-oxidation agent and the properties of the starting α-alkenoic acid or ester but is typically in the range of about 0.5% to 5% on a volume-to-volume basis with the volume of the second reaction medium. Oxygenation must be maintained during this bio-oxidation phase and can be accomplished by vigorous agitation of the reaction mixture, by supplying compressed air, or by running the bio-oxidation phase in an oxygen enriched atmosphere. The pH of the reaction mixture during the bio-oxidation phase is maintained between 6.5 and 7.5 by the addition of a suitable base such as aqueous sodium hydroxide. The temperature of the reaction mixture during the bio-oxidation phase is between 2° and 40° C. The length of the bio-oxidation phase will vary depending upon the bio-oxidation agent and the properties of the starting α-alkenoic acid or ester from 12 hours to 7 days.

### Product Isolation

The product of Formula I may be isolated in some instances by first treating the reaction mixture with a suitable acid such as sulfuric acid or hydrochloric acid. This acidified mixture is then extracted with a suitable solvent such as ethyl acetate, hexane, heptane, long chain fatty alcohols (carbon number >4), long chain ketones (carbon number >4), long chain aldehydes (carbon number >4), or some combination of these solvents at elevated temperature. The organic extracts are then allowed to cool and the desired products isolated by standard techniques, including crystallization and chromatography. Alternatively, the reaction mixture may be extracted with a suitable solvent as described above without prior acidification of the reaction mixture.

### Ester Cleavage Reaction

The skilled artisan will appreciate that compounds of Formula I and II where R is other than hydrogen are esters. During the present bio-oxidation process, the ester moiety may be cleaved to provide compounds of Formula I where R is H depending on the specific reaction conditions and the structure of the substrate of Formula II. When this occurs, and if the resultant dicarboxylic acid of Formula I is the desired product, then that dicarboxylic acid product may be isolated from the reaction mixture with or without further purification as necessary or desired.

Further, there are instances where the ester moiety of a starting α-alkenoic ester is not cleaved during the bio-oxidation process of Scheme 1, requiring further steps to be taken subsequent to the bio-oxidation should an α,ω-alkanedioic acid product be desired.

This process is illustrated below in Scheme 1 comprising an ester cleavage as illustrated in Scheme 2 where variables “R′” and “n” are previously defined herein.

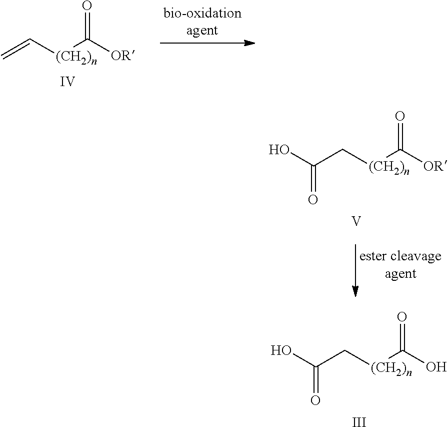

In an embodiment, bio-oxidation is performed on the starting α-alkenoic ester of Formula IV as previously described to provide the ester product of Formula V. Methods for cleaving the ester moiety of Formula V to provide the α,ω-alkanedioic acid of Formula III are well known to the skilled artisan and are described in the general reference texts identified above. Specific conditions are described in Greene at pages 699-812, as well as C. J. Salomon, et al., Tetrahedron, 49, 3691(1993). The skilled person will appreciate that the ester cleavage step may be performed in situ in the bio-oxidation reaction mixture or after isolation of the ester product, with or without further purification, in a subsequent reaction. The final α,ω-alkanedioic acid of Formula III may be isolated and purified by standard techniques well known to the skilled artisan including extraction, evaporation, precipitation, chromatography, filtration, trituration, and crystallization.

In certain instances the ester moiety of a compound of Formula V may be enzymatically cleaved to provide the α,ω-alkanedioic acid of Formula III. In an embodiment, bio-oxidation of the starting α-alkenoic ester of Formula IV described herein provides the ester product of Formula V, followed by an enzymatic cleavage of the ester moiety of Formula V to provide the α,ω-alkanedioic acid of Formula III. Methods for enzymatic cleavage of esters are well known to the skilled artisan. K. Faber and S. Riva, Synthesis, 895 (1992); H. Waldmann and D. Sebastian, Chemical Reviews, 94, 911 (1994); Enzyme Catalysis in Organic Synthesis: A Comprehensive Handbook, Edited by K. Drauz and H. Waldmann, VCH, New York (1995).

Specific conditions for the enzymatic cleavage of heptyl, 2-N-(morpholino)ethyl, choline, (methoxyethoxy)ethyl, and methoxyethyl esters are described in Greene at pages 711-713. Enzymes useful for this enzymatic cleavage include lipases from Rhizopus niveus, Aspergillis niger, and Mucor javanicus, as well as Newlase F and butyrylcholine esterase and are either commercially available or may be isolated by methods well known to the skilled artisan. The skilled person will appreciate that the ester cleavage step may be performed in situ in the bio-oxidation reaction mixture or after isolation of the ester product, with or without further purification, in a subsequent reaction. The final α,ω-alkanedioic acid of Formula III may be isolated and purified by standard techniques well known to the skilled artisan.

The ester moiety of the starting α-alkenoic ester of Formula IV is cleaved prior to bioxidation to provide the α-alkenoic acid of Formula VI under the conditions described herein and as illustrated in the following scheme where “R′” and “n” are as previously defined:

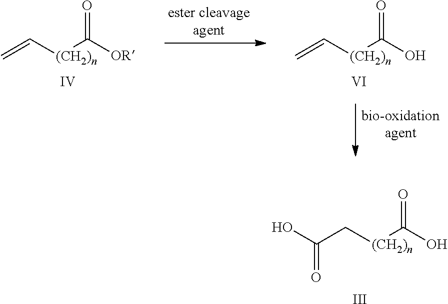

The α-alkenoic acid of Formula VI can be isolated prior to the subsequent bio-oxidation step or used directly in the bio-oxidation step without further purification. Alternatively, the alcohol product, R′—OH resulting from the ester cleavage step may be removed from the reaction mixture prior to the bio-oxidation step if necessary or desired. The final α,ω-alkanedioic acid of Formula III may be isolated and purified by standard techniques.

### ADDITIONAL EMBODIMENTS

Embodiment 1. A method of preparing α,ω-alkanedioic acids or esters of Formula I:

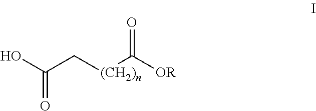

where n≥1 and R is H, C₁-C₁₈ alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl comprising reacting a compound of Formula II.

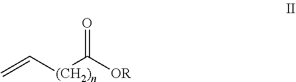

where n≥1 and R is H, C₁-C₈ alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl with a suitable bio-oxidation agent.

Embodiment 2. A method of Embodiment 1 where the bio-oxidation agent is a yeast, bacteria, or filamentous fungus, or a combination thereof.

Embodiment 3. A method of any of Embodiments 1-2 where the bio-oxidation agent is a yeast.

Embodiment 4. A method of any of Embodiments 1-3 where the bio-oxidation agent is one or more naturally occurring or modified yeast including Saccharomyces cerevisae, Yarrowia lipolytica, Yarrowia galli, Yarrowia oslonensis, Scheffersomyces stipitis, Candida parapsilosis, Candida maltosa, Candida viswanathii, Candida psuedoglaebosa, Meyerozyma guilliermondii, Debaryomyces hansenii, Candida tropicalis, Lodderomyces. elongisporus, Diutina catenulata, Yarrowia deformans, Candida phangneansis, Kodamaea ohmeri, Starmerella apicola, Starmerella bombicola, Candida krusei, Pichia fermentans, Candida alimentaria, Candida hispaniensis, and Candida hollandica.

Embodiment 5. A method of any of Embodiments 1-4 where the bio-oxidation agent is one or more naturally occurring or modified yeast selected from Yarrowia lipolytica, Candida maltosa, Candida viswanathii, Candida tropicalis, and Candida hollandica.

Embodiment 6. A method of any of Embodiments 1-5 where n≥7.

Embodiment 7. A method of any of Embodiments 1-6 where n=7-9.

Embodiment 8. A method of any of Embodiments 1-7 where R is H or C₁-C₄ alkyl.

Embodiment 9. A method of any of Embodiments 1-8 where R is H, methyl, or ethyl.

Embodiment 10. A method of any of Embodiments 1-9 where R is H, heptyl, 2-N-(morpholinyl)ethyl, choline, (methoxyethoxy)ethyl, or methoxyethyl.

Embodiment 11. A method of any of Embodiments 1-10 where R is H.

Embodiment 12. A method of any of Embodiments 1-11 where R is heptyl or methyl.

Embodiment 13. A method of preparing α,ω-alkanedioic acids of Formula III:

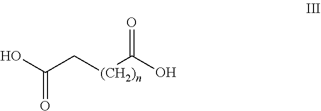

where n≥1 comprising:
a) reacting a compound of Formula IV:

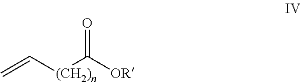

where n≥1 and R′ is C₁-C₁₈ alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl with a suitable bio-oxidation agent, to provide a compound of Formula V

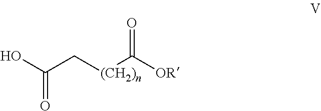

and then
b) cleaving the ester moiety.

Embodiment 14. A method of Embodiment 13 where the bio-oxidation agent is a yeast, bacteria, or filamentous fungus, or a combination thereof.

Embodiment 15. A method of any of Embodiments 13-14 where the bio-oxidation agent is a yeast.

Embodiment 16. A method of any of Embodiments 13-15 where the bio-oxidation agent is one or more naturally occurring or modified yeast including Saccharomyces cerevisae, Yarrowia lipolytica, Yarrowia galli, Yarrowia oslonensis, Scheffersomyces stipitis, Candida parapsilosis, Candida maltosa, Candida viswanathii, Candida psuedoglaebosa, Meyerozyma guilliermondii, Debaryomyces hansenii, Candida tropicalis, Lodderomyces. elongisporus, Diutina catenulata, Yarrowia deformans, Candida phangneansis, Kodamaea ohmeri, Starmerella apicola, Starmerella bombicola, Candida krusei, Pichia fermentans, Candida alimentaria, Candida hispaniensis, and Candida hollandica.

Embodiment 17. A method of any of Embodiments 13-16 where the bio-oxidation agent is one or more naturally occurring or modified yeast selected from Yarrowia lipolytica, Candida maltosa, Candida viswanathii, Candida tropicalis, and Candida hollandica.

Embodiment 18. A method of any of Embodiments 13-17 where n≥7.

Embodiment 19. A method of any of Embodiments 13-18 where n=7-9.

Embodiment 20. A method of any of Embodiments 13-19 where R′ is C₁-C₈ alkyl, 2-N-(morpholinyl)ethyl, choline, (methoxyethoxy)ethyl, or methoxyethyl.

Embodiment 21. A method of any of Embodiments 13-20 where R′ is methyl, or ethyl.

Embodiment 22. A method of any of Embodiments 13-21 where R′ is heptyl, 2-N-(morpholinyl)ethyl, choline, (methoxyethoxy)ethyl, or methoxyethyl.

Embodiment 23. The method of Embodiment 13, wherein the ester moiety is cleaved by chemical hydrolysis or by an enzyme, wherein the enzyme is added in an in vitro enzymatic hydrolysis or expressed by a microorganism in vivo.

Embodiment 24. A method of Embodiment 23 where the enzyme is Newlase F, butyrylcholine esterase or a lipase from Rhizopus niveus, Aspergillis niger, or Mucor javanicus.

Embodiment 25. The method of preparing α,ω-alkanedioic acids of Formula III:

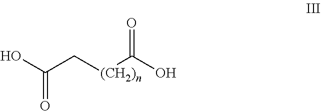

where n≥1 comprising:
a) reacting a compound of Formula IV:

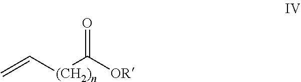

where n≥1 and R′ is C₁-Cis alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl with an ester cleavage agent, to provide a compound of Formula VI:

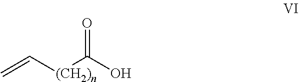

and then
b) reacting the compound of Formula VI with a bio-oxidation agent.

### PREPARATION AND EXAMPLES

The features of the disclosure are described in the following non-limiting preparations examples.

### Preparation 1

### Buffered Glycerol-Complex Medium (“BMGY”)

Dissolve 10 g yeast extract and 20 g peptone in 700 mL of water. Autoclave this solution for 20 minutes on liquid cycle. Cool this solution to room temperature and then add 100 mL of 1 M potassium phosphate buffer (pH 6.0), 100 mL 10× Yeast Nitrogen Base (1.34%), 20 mL 500× biotin (0.00004%), and 100 mL 10× glycerol (1%) and mix well.

### Preparation 2

### Buffered Methanol-Complex Medium (“BMMY”)

Dissolve 10 g yeast extract and 20 g peptone in 700 mL of water. Autoclave this solution for 20 minutes on liquid cycle. Cool this solution to room temperature and then add 100 mL of 1 M potassium phosphate buffer (pH 6.0), 100 mL 10× Yeast Nitrogen Base (1.34%), 20 mL 500× biotin (0.00004%), and 100 mL 10× methanol (0.5%) and mix well.

### Example 1

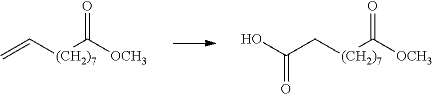

BMGY medium (3 mL) was inoculated with Candida visavanathii (ATCC@20962™) and the seed culture was allowed to grow overnight at 30° C. BMGY medium (10 mL) containing 0.1 mL methyl 9-decenoate was then inoculated with 0.2 mL of the seed culture and this mixture was vigorously agitated at 30° C. for 3 days. The reaction mixture was then extracted with ethyl acetate. The ethyl acetate phases were collected and concentrated under reduced pressure. The residue was dissolved in 4:1 water: acetonitrile and analyzed as described below. Methyl 1,9-decandioic acid was identified from the reaction mixture. MS(m/z)=216.14.

### Example 2

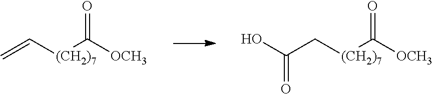

The procedure of Example 1 was performed starting with methyl 9-decenoate as described above, except that the bio-oxidation reaction time was reduced from 3 days to 12 hours to provide methyl 1,9-decanedioic acid from the reaction mixture. MS(m/z)=216.14 (Exact Mass).

### Example 3

### LC-MS Results of Overnight Bioconversion of Different Feeds

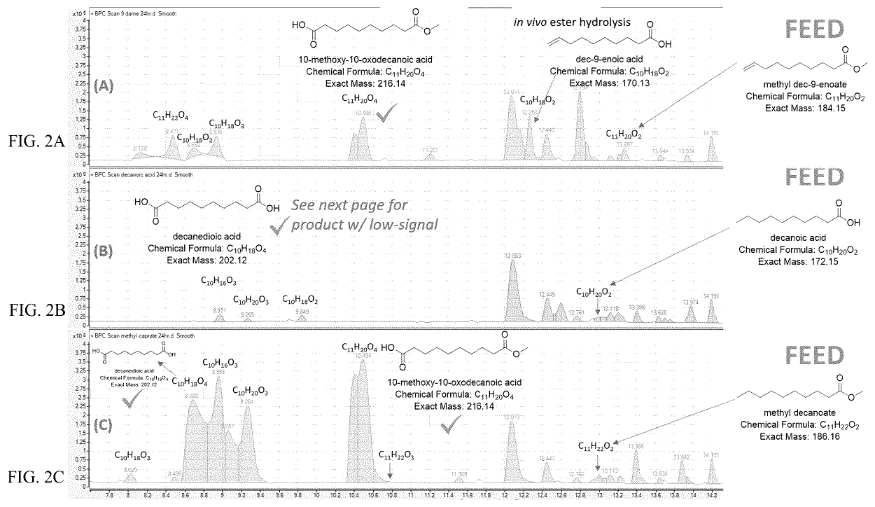

As described above, our data has shown that bioconversion works. As shown in FIG. 2A, the end product 10-methoxy-10-oxodecanoic acid is detected. Sebacic acid (“SA”) can be obtained from the ester cleavage of the 10-methoxy-10-oxodecanoic acid. Note, in vivo ester hydrolysis by the tested yeast was observed. As shown in FIG. 2B, the end product SA and intermediate were detected during the bioconversion of decanoic acid. As shown in FIG. 2C, both intermediate 10-methoxy-10-oxodecanoic acid and desired end product SA were detected. Note: in vivo ester hydrolysis by the tested yeast was observed. Furthermore, the yeast strain used in this round of test was the same, Candida viswanathii Sandhu et Randhawa (ATCC® 20962™). The conversion period was shortened to be overnight (˜12 hours).

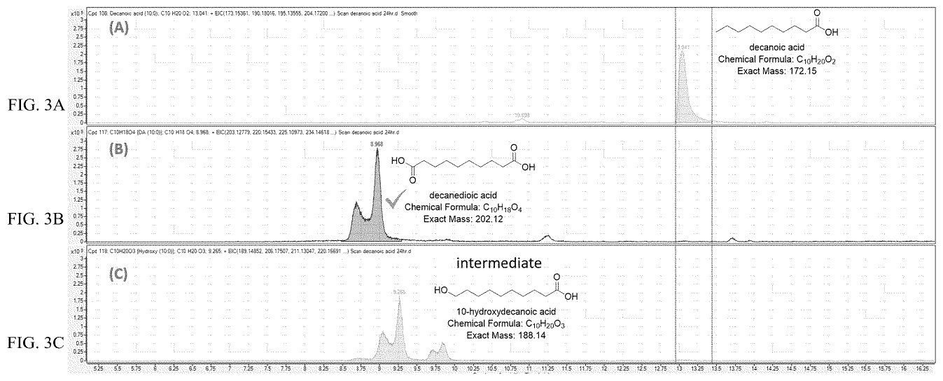

As described above, our data has shown that bioconversion works. FIG. 3A shows the extracted ion chromatograph data of a bioconversion of decanoic acid. FIG. 3B shows the extracted ion chromatograph for the product decanedioic acid. FIG. 3C shows the extracted ion chromatograph for the intermediate molecule 10-hydroxydecanoic acid.

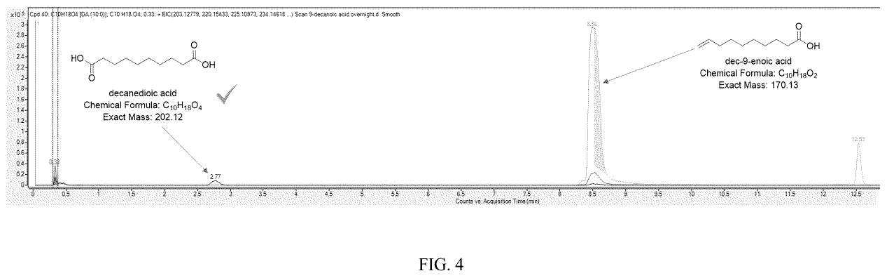

In the bioconversion of dec-9-enoic acid (FIG. 4), the yeast strain used in this round of test was the same, Candida viswanathii Sandhu et Randhawa (ATCC® 20962™). The conversion period was shortened to be overnight (˜12 hours). BPC means base peak chromatogram and extracts the largest ion from the total ion chromatogram (TIC). EIC means extracted ion chromatogram.

Liquid Chromatography Coupled with Tandem Mass Spectrometry Analysis and ELSD

Agilent 1260 liquid chromatography system equipped with Poroshell 120 EC-C18 column (2.7 μm, 2.1 mm×150 mm) was used for the liquid chromatography analysis of samples. The temperature of the column was set at 30° C., and the flow rate of the mobile phase was 0.5 mL/min. Water (A) and acetonitrile (B) were used as the mobile phase. The gradient conditions during the LC analysis were as follows: 0-0.5 min, 20% B; 0.5-7 min, ramping from 20 to 70% B; 7-12 min, ramping from 70 to 100% B; 12-14 min, hold at 100% B; 14-15 min, ramping down to 20% B; post-run hold at 20% B for 2 min.

The system is equipped with a 1:1 splitter post column directing sample flow to two detectors: Agilent 1260 Infinity II ELSD (set to 30° C. with nebulizer gas flow rate of 1.59 L/min) and Agilent 6546 Q-TOF equipped with Agilent Jet Stream (AJS) source set for negative ionization. Source settings for AJS were as follows: Gas Temp 200° C., Drying Gas 12 L/min, Nebulizer 35 psi, Sheath Gas Temp 325° C., and Sheath Gas flow 12 L/min. Acquisition settings for Q-TOF were as follows: 50-1000 m/z range, 4 spectra per second MS acquisition rate for MS mode. For MS/MS analysis, MS acquisition rate of 6 spectra per second and MS/MS acquisition rate of 2 spectra per second were set.

Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

1. A method of preparing α,ω-alkanedioic acids or esters of Formula I: 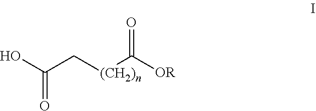
where n≥1 and R is H, C₁-C₁₈ alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl comprising reacting a compound of Formula II: 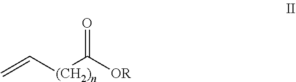
where n≥1 and R is H, C₁-C₈ alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl with a suitable bio-oxidation agent.

2. The method of claim 1, wherein the bio-oxidation agent is a yeast, bacteria, or filamentous fungus, or a combination thereof.

3. The method of claim 2, wherein the bio-oxidation agent is a yeast.

4. The method of claim 1, where the bio-oxidation agent is one or more naturally occurring or modified yeast including Saccharomyces cerevisae, Yarrowia lipolytica, Yarrowia galli, Yarrowia oslonensis, Scheffersomyces stipitis, Candida parapsilosis, Candida maltosa, Candida viswanathii, Candida psuedoglaebosa, Meyerozyma guilliermondii, Debaryomyces hansenii, Candida tropicalis, Lodderomyces. elongisporus, Diutina catenulata, Yarrowia deformans, Candida phangneansis, Kodamaea ohmeri, Starmerella apicola, Starmerella bombicola, Candida krusei, Pichia fermentans, Candida alimentaria, Candida hispaniensis, and Candida hollandica.

5. The method of claim 1, wherein n≥7.

6. The method of claim 1, wherein n=7-9.

7. The method of claim 1, wherein R is H, C₁-C₄ alkyl, heptyl, methyl, or ethyl.

8. The method of claim 1, wherein R is H, heptyl, 2-N-(morpholinyl)ethyl, choline, (methoxyethoxy)ethyl, or methoxyethyl.

9. A method of preparing α,ω-alkanedioic acids of Formula III: 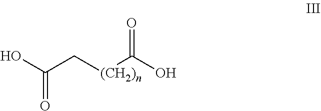
where n≥1 comprising:
a) reacting a compound of Formula IV: 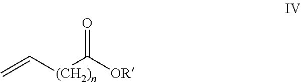
where n≥1 and R′ is C₁-C₁₈ alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl with a bio-oxidation agent, to provide a compound of Formula V 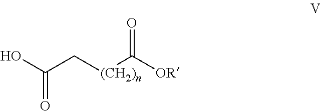
and then
b) cleaving the ester moiety.

10. The method of claim 9, wherein the bio-oxidation agent is a yeast, bacteria, or filamentous fungus, or a combination thereof.

11. The method of claim 9, wherein the bio-oxidation agent is a yeast.

12. The method of claim 9, wherein the bio-oxidation agent is one or more naturally occurring or modified yeast including Saccharomyces cerevisae, Yarrowia lipolytica, Yarrowia galli, Yarrowia oslonensis, Scheffersomyces stipitis, Candida parapsilosis, Candida maltosa, Candida viswanathii, Candida psuedoglaebosa, Meyerozyma guilliermondii, Debaryomyces hansenii, Candida tropicalis, Lodderomyces. elongisporus, Diutina catenulata, Yarrowia deformans, Candida phangneansis, Kodamaea ohmeri, Starmerella apicola, Starmerella bombicola, Candida krusei, Pichia fermentans, Candida alimentaria, Candida hispaniensis, and Candida hollandica.

13. The method of claim 9, wherein n≥7.

14. The method of claim 9, wherein n=7-9.

15. The method of claim 9, wherein R′ is C₁-C₈ alkyl, 2-N-(morpholinyl)ethyl, choline, (methoxyethoxy)ethyl, methoxyethyl, heptyl, methyl, or ethyl.

16. The method of claim 9, wherein the ester moiety is cleaved by chemical hydrolysis or by an enzyme, wherein the enzyme is added in an in vitro enzymatic hydrolysis or expressed by a microorganism in vivo, wherein the enzyme is Newlase F, butyrylcholine esterase or a lipase from Rhizopus niveus, Aspergillis niger, or Mucor javanicus.

17. A vertically-integrated process for producing 1-decene and sebacic acid comprising the steps of:
producing a fatty acid methyl ester from vegetable oil fatty acid;
combining a ruthenium catalyst with ethylene and the fatty acid methyl ester to produce 1-decene and 9-decenoic acid methyl ester (9-DAME); and
separating 9-DAME from 1-decene; and
converting 9-DAME with a bio-oxidation agent to produce sebacic acid and methanol wherein methanol is recycled and/or reused.

18. A method of preparing α,ω-alkanedioic acids of Formula III: 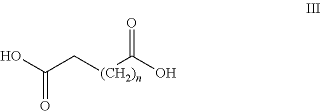
where n≥1 comprising:
a) reacting a compound of Formula IV: 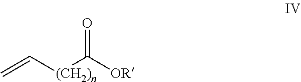
where n≥1 and R′ is C₁-Cis alkyl, optionally substituted C₂-C₅ alkenyl, C₃-C₇ cycloalkyl, acetol, 2-N-(morpholinyl)ethyl, (methoxyethoxy)ethyl, methoxyethyl, choline, substituted methyl, 2-substituted ethyl, optionally substituted phenyl, optionally substituted benzyl, optionally substituted phenacyl, or silyl with an ester cleavage agent, to provide a compound of Formula VI: 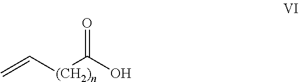
and then
b) reacting the compound of Formula VI with a bio-oxidation agent.
